(19) United States
(12) Patent Application Publication
(10) Pub. No.: US 2011/0015741 A1
(43) Pub. Date: Jan. 20, 2011

(54) SPINAL IMPLANT CONFIGURED TO APPLY RADIATION TREATMENT AND METHOD

(75) Inventors: Anthony J. Melkent, Memphis, TN (US); Dianna S. Parimore, Arlington, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/504,290

(22) Filed: Jul. 16, 2009

(51) Int. Cl. A61F 2/44; A61N 5/00

(52) U.S. Cl. 623/17.11; 623/17.16; 600/3

(57) ABSTRACT

Embodiments of the invention include a vertebral implant configured to replace at least a portion of a central vertebra and to direct therapeutic radiation toward at least a treatable portion of tissue. The treatable portion of tissue may include one or more adjacent treatable vertebra.

### FIELD OF THE INVENTION

The present invention relates generally to the field of one or both of replacing and supporting portions of the human structural anatomy with medical implants and optionally applying radiation treatment to selected adjacent tissues. The present invention more particularly relates to one or both of replacing and supporting at least a portion of a vertebra and optionally applying radiation treatment to tissues of a spinal column.

### BACKGROUND

It is sometimes necessary to remove one or more vertebrae or a portion of the one or more vertebrae from the human spine in response to various pathologies. For example, one or more vertebrae may become damaged as a result of tumor growth. Removal, or excision, of a vertebra may be referred to as a vertebrectomy. Excision of a generally anterior portion of a vertebra, or vertebral body, may be referred to as a corpectomy. An implant is usually placed between the remaining vertebrae to provide structural support for the spine as a part of a corpectomy or vertebrectomy. FIG. 1 illustrates four vertebrae, V₁-V₄ of a typical lumbar spine and three spinal discs, D₁-D₃. As illustrated, V₃ is a damaged vertebra and all or a part of V₃ could be removed to help stabilize the spine. If removed along with spinal discs D₂ and D₃, an implant may be placed between vertebrae V₂ and V₄. In some cases, one or both of V₂ and V₄ may be known to include, or may be suspected of including, unwanted tissue such as cancerous cells. The implant inserted between the vertebrae may be designed to facilitate fusion or to provide spinal stability between remaining vertebrae. A successful procedure may decrease pain, preserve or enhance neurological function, and allow a patient greater mobility without an external orthosis. All or part of more than one vertebra may be damaged and require removal and replacement in some circumstances. If only a portion of a vertebral body and adjacent discs are removed and replaced, the procedure may be called a hemi-vertebrectomy.

An improved corpectomy, vertebrectomy, hemi-vertebrectomy, or other vertebral implant may direct radiation toward adjacent spinal tissues where tissues that are known or suspected of including one or more of cancerous cells and tumors are located. Some improved devices may include one or more caps that connect to a conventional strut and direct radiation from the one or more caps in particular directions. In some cases, it may be advantageous to be able to selectively alter one or more of the intensity and the direction of radiation transmitted from a vertebral implant.

### SUMMARY

One embodiment of the invention is a vertebral implant for replacing at least an anterior portion of a central vertebra and at least portions of two spinal discs, wherein at least an anterior portion of the central vertebra is removed from between a first vertebra and a second vertebra. The vertebral implant may include a strut having a first end and an opposite second end and a first cap coupled to the first end of the strut. The first cap may include a proximal end coupled to the first end of the strut, a distal end configured to engage with the first vertebra, a first wall extending between the proximal end of the first cap and the distal end of the first cap, wherein the first wall includes materials that substantially block the transmission of radiation through the first wall, and a first floor extending from the first wall. In some embodiments, the first floor and the first wall form a cavity near the distal end of the first cap. The first floor may include materials that substantially block the transmission of radiation through the first floor toward the proximal end of the first cap. The vertebral implant may be adapted or sized to contact the first vertebra and the second vertebra and provide support between the first vertebra and the second vertebra.

An embodiment of the invention is a means for applying radiation to a vertebral body. The embodiment may include a spacing means for providing support between two vertebral bodies and a radiation emitting device coupled to the spacing means. The spacing means may include a radiation containment means that is capable of blocking transmission of radiation from the radiation emitting device in one or more directions and permitting transmission of radiation from the radiation emitting device in one or more directions.

Another embodiment of the invention is a method of irradiating cells of a treatable vertebral body in a spinal column. The method embodiment may include removing a central vertebral body that is adjacent to the treatable vertebral body. The method embodiment may also include providing a vertebral body replacement implant with a cavity. The vertebral body replacement implant may substantially block the transmission of radiation in all directions except the direction toward the treatable vertebral body when the vertebral body replacement implant is implanted. Method embodiments may also include inserting a radiation emitting device in the cavity in the vertebral body replacement implant, and implanting the vertebral body replacement implant in a spinal column.

### BRIEF DESCRIPTION OF THE DRAWINGS

FIG. 1 is an elevation view of a segment of a lumbar spine.

FIG. 2 is a perspective view of an embodiment of a vertebral implant.

FIG. 3 is a cross-sectional view through a portion of the vertebral implant of FIG. 2.

FIG. 4 is a perspective view of portions of an embodiment of a vertebral implant and an instrument that may be used to implant portions of the vertebral implant.

FIG. 5 is a partially exploded perspective view of an embodiment of a vertebral implant in a substantially contracted state.

FIG. 6 is a perspective view of an embodiment of a vertebral implant in a substantially expanded state, and an instrument that may be used to insert and expand the vertebral implant.

FIG. 7 is a partially exploded perspective view of an embodiment of a vertebral implant.

FIG. 8 is a plan view of a component of the vertebral implant of FIG. 7.

### DETAILED DESCRIPTION

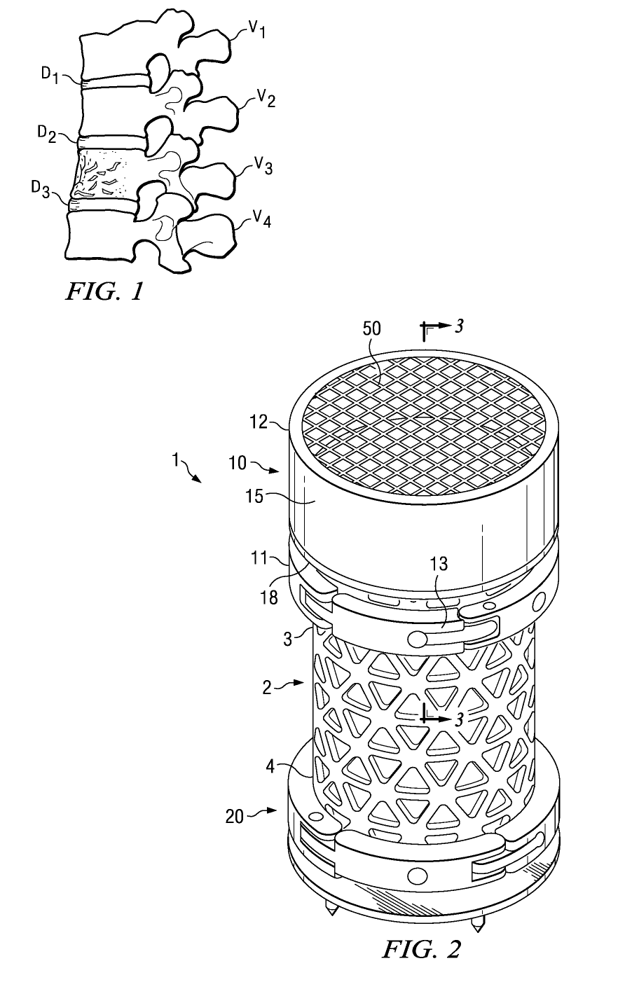

FIGS. 2-8 illustrate various embodiments of a vertebral implant 1, 101, 201, 301 for replacing at least an anterior portion of a vertebra. For example and without limitation, the vertebral implant 1, 101, 201, 301 may be employed to replace all or an anterior portion of the damaged vertebra V₃ shown in FIG. 1, which is illustrated as a central vertebra between two vertebrae V₂ and V₄. Similarly, the vertebral implant 1, 101, 201, 301, or another functional vertebral implant within the scope of the present disclosure, may be used to replace anterior portions of V₂ and V₃, or any other combination of vertebrae. Embodiments of the vertebral implant 1, 101, 201, 301 may be, without limitation, corpectomy, vertebrectomy, or hemi-vertebrectomy devices, as defined with reference to FIG. 1. Any number of vertebrae that are at least partially removed may singly or in combination include a “central vertebra” as used herein. Discs adjacent to removed or partially removed vertebrae may be partially or completely removed prior to placement of an embodiment of a vertebral implant 1, 101, 201, 301.

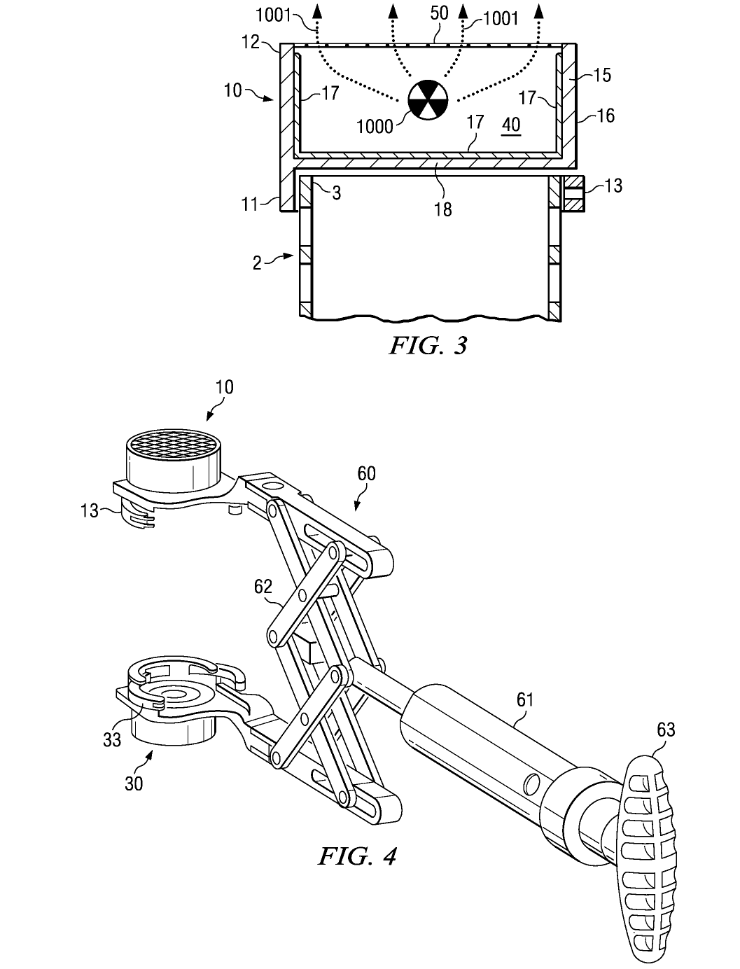

The vertebral implant 1 is illustrated in FIGS. 2-3, and in part in FIG. 4, and includes a strut 2 and a first cap 10. The vertebral implant 1 may be sized to contact a first vertebra adjacent to a space left by one or more central vertebra removed from a spinal column, and also sized to contact a second vertebra adjacent to the space left by one or more central vertebra removed from a spinal column. The vertebral implant 1 may be sized to provide support between the first vertebra and the second vertebra.

The strut 2 includes a first end 3 and a second end 4. The strut 2 illustrated includes a section of PYRAMESH® Surgical Titanium Mesh available from Medtronic, Inc. The mesh of strut 2 is shown with generally triangular openings. In other embodiments, the strut 2 may include holes of any shape, slots, or may not include openings. In addition or as an alternative to titanium, the strut 2 may be made from any biocompatible material. For example and without limitation, the strut 2 may be made in whole or in part from polyetheretherketone (PEEK), cobalt chrome, stainless steel, and any biocompatible metal, metal alloy, or polymer. The strut 2 may also include a bone or bone-based material. For example and without limitation, the strut 2 may include in whole or in part one or more of allograft, xenograft, demineralized bone, and autograft. In some embodiments, the strut 2 is an expandable strut configured to expand from a first height of a second taller height.

The first cap 10 of the illustrated embodiment is coupled to the first end 3 of the strut 2 at a proximal end 11 of the first cap 10. In some embodiments, a first cap may be integral with a strut to form a monolithic body that in whole or in part forms the vertebral implant. In the embodiment shown, the first end 3 of the strut 2 is captured behind a gate 13 at the proximal end 11 of the first cap 10. By way of non-limiting example, the gate 13 may include a gate mechanism such as the gate mechanism of a SCEPTOR™ Universal Endcleat available from Medtronic, Inc. The first cap 10 shown includes a first wall 15 extending between the proximal end 11 of the first cap 10 and the distal end 12 of the first cap 10. The illustrated first wall 15 is a substantially circular wall in cross-section that forms a continuous lateral enclosure. In other embodiments, a first wall may be formed in combination with other walls or wall components or singularly form a full or partial lateral enclosure of any functional shape. For example and without limitation, the lateral periphery of one or more walls may be substantially an oval, kidney shape, triangle, rectangle, square, any polygonal or curved shape, or any combination of shapes. Embodiments of the first wall include materials that substantially block the transmission or radiation through the first wall.

The first wall 15 illustrated in FIG. 3 in cross-section includes a structural component 16 and a radiation blocking material 17. The structural component 16 of various embodiments may also be capable of blocking the transmission of radiation. As used herein, the term “blocking the transmission of radiation” and similar terms mean that a component blocks the passage of therapeutically effective amounts of radiation from a radiation emitting device. The blocking of radiation may not be complete such that there is no measurable amount of radiation allowed through a component. The structural component 16 of some embodiments does not significantly block the transmission of radiation and may in these embodiments be referred to as a radiolucent material. For example and without limitation, a radiolucent structural component 16 may be made from PEEK material. The first wall 15 may be made from a material that substantially blocks the transmission of radiation through the first wall 15. Non-limiting example materials of which the first wall 15 may be made that block the transmission of radiation include cobalt chrome, titanium, and stainless steel. A radiation blocking material may be applied to the inside or outside or be encapsulated within a wall such as the first wall 15. As shown in FIG. 3, a radiation blocking material 17 is applied to the inside of the first wall 15. Materials of which the radiation blocking material 17 may be made or may include are, by way of example and without limitation, gold, lead, barium, bismuth, tantalum, tin, and tungsten. A radiation blocking material may be applied to or integrated with a wall embodiment by any effective mechanism, including but not limited to, chemically bonding, an intervening adhesive, welding, melting, press fitting, ion deposition, or mechanically locking.

As shown in FIGS. 2 and 3, a first floor 18 extends from the first wall 15. The illustrated first wall 15 and the first floor 18 form a cavity 40 near the distal end 12 of the first cap 10. In the illustrated embodiment, the first wall 15 forms a continuous lateral enclosure and the first floor 18 connects with first wall 15 around the entire periphery of the first cap 10. In other embodiments, the first floor 18 may connect with the first wall 15 in part and with other components or walls in part. The first floor 18 of the illustrated embodiment blocks the transmission of radiation through the first floor 18 toward the proximal end 11 of the first cap 10. In other embodiments, a first floor may be designed to allow the transmission of a selected amount of radiation through the first floor. The first floor 18 may be similar to the wall 15 wherein it is made from materials that block the transmission of radiation alone, or the first floor 18 may comprise a structural component in combination with a radiation blocking material such as the radiation blocking material 17.

The vertebral implant 1 illustrated in FIGS. 2 and 3 includes a ceiling 50 extending across at least a portion of the distal end 12 of the first cap 10. The ceiling 50 shown is a mesh of strands that extend transversely to one another to form the ceiling 50. The illustrated mesh of strands may be either constructed from material that blocks the transmission of radiation or from radiolucent material. In the first instance, radiation would be permitted to pass from the distal end 12 between the mesh strands, or in the second instance, radiation would be permitted to pass from the distal end 12 both through and between the mesh strands.

In some embodiments, a ceiling is configured to support load transferred from an endplate of the first vertebra to the first cap 10. The ceiling 50, for example, would be configured to support load transferred from an endplate of the first vertebra when the strands of the mesh are thick enough to provide resistance in bending and the connections to the first wall 15 of the ceiling 50 are sufficient to support loading, rather than merely to be deflected and to allow substantially all of the load to bear on the first wall 15.

The vertebral implant 1 may also include a radiation emitting device. A radiation emitting device 1000 is illustrated in FIG. 3 within the cavity 40. In other embodiments, a radiation emitting device may be located at any effective location within, near, or on a vertebral implant. For example and without limitation, a radiation emitting device may be along the length of the device. The direction of radiation transmission may be in any effective direction, including from the side of a vertebral implant, or a combination of from one or both ends of an implant and from the side of the vertebral implant. The radiation emitting device 1000 is shown in FIG. 3 projecting radiation 1001, depicted by arrows, from the distal end 12 of the first cap 10. The radiation emitting device 1000 may include any therapeutically effective radiation source. Suitable radiation sources for use in the radiation emitting device 1000 of the vertebral implant 1 of some embodiments include both solids and liquids. By way of non-limiting example, the radiation source may be a radionuclide, such as I-125, I-131, Yb-169, Ir-192 or other radionuclides that emit photons, beta particles, gamma radiation, or other therapeutic energy or substances. The radioactive material may also be a fluid made from any solution of radionuclide(s), e.g., a solution of I-125 or I-131, or a radioactive mixture may be produced using a slurry of a suitable fluid containing small particles of solid radionuclides, such as Au-198, Y-90. Radionuclides may also be delivered in a gel. One radioactive material useful in some embodiments is Iotrex®, a nontoxic, water soluble, nonpyrogenic solution containing sodium 3-(125I)iodo-4-hydroxybenzenesulfonate (125I-HBS), available from Proxima Therapeutics, Inc. of Alpharetta, Ga. Radioactive micro spheres of the type available from the 3M Company of St. Paul, Minn., may also be used. A radioactive source may be preloaded into a vertebral implant, catheter, or other vessel at the time of manufacture or loaded after the vertebral implant, catheter, or other vessel has been implanted. By way of further non-limiting example, one or more solid radioactive micro spheres may be inserted through a catheter on a wire and into or adjacent the vertebral implant.

An insertion instrument 60 is illustrated in FIG. 4 in a partially expanded state with the first cap 10 and a second cap 30 positioned in the insertion instrument 60. The insertion instrument 60 includes a central body 61, a linkage 62, and a handle 63. In operation, the first and second caps 10, 30 are introduced into an operative field with the insertion instrument 60. The central body 61 of the insertion instrument 60 may be grasped by a user or by another instrument and the handle 63 turned relative to the central body 61 to actuate the linkage 62 and move the first cap 10 and the second cap 30 closer together or farther apart. When expanded such that the first cap 10 and the second cap 30 are positioned against respective vertebrae, a strut, such as the strut 2 described in detail herein, or any functionally sufficient strut, may be inserted between the first cap 10 and the second cap 30. A strut may be inserted through the gate 13 and a gate 33, and then the gates 13, 33 may be closed to capture the strut within the first and second caps 10, 30. The insertion instrument 60 may then be contracted and removed from the operative field.

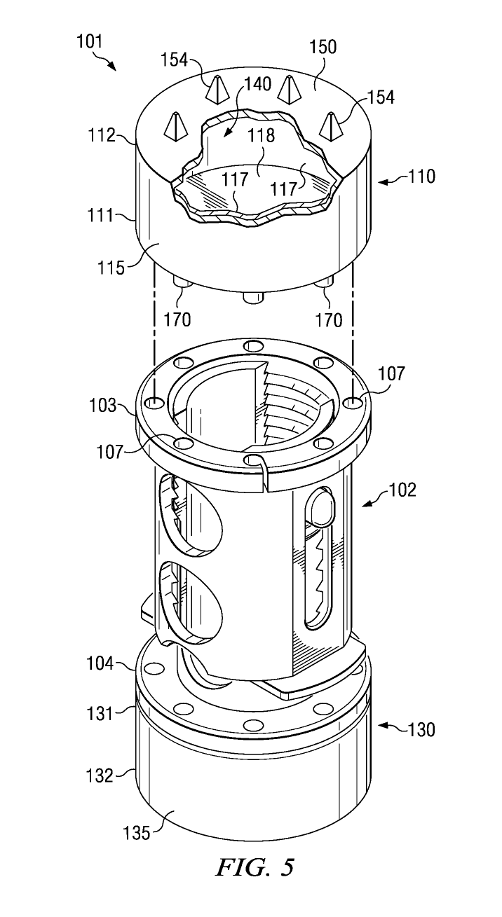

The vertebral implant 101 is illustrated in FIG. 5, and includes an expandable strut 102, a first cap 110, and a second cap 130. The illustrated first cap 110 is shown in a partially cut-away view to better illustrate some components. The vertebral implant 101 may be sized to contact a first vertebra adjacent to a space left by one or more central vertebra removed from a spinal column, and also sized to contact a second vertebra adjacent to the space left by one or more central vertebra removed from a spinal column. The vertebral implant 101 may be sized to provide support between the first vertebra and the second vertebra.

The strut 102 includes a first end 103 and a second end 104. The expandable strut 102 is illustrated in a contracted state with portions of the second end 104 concentrically nested within the first end 103. The expandable strut 102 may include titanium material and may be expandable by ratcheting between components of the first and second ends 103, 104. In addition or as an alternative to titanium, the expandable strut 102 may be made from any biocompatible material. For example and without limitation, the expandable strut 102 may be made in whole or in part from polyetheretherketone (PEEK), cobalt chrome, stainless steel, and any biocompatible metal, metal alloy, or polymer. The expandable strut 102 may also include a bone or bone-based material. For example and without limitation, the expandable strut 102 may include in whole or in part one or more of allograft, xenograft, demineralized bone, and autograft.

The first cap 110 of the illustrated embodiment is shown in an exploded position relative to the expandable strut 102 and aligned to be coupled to the first end 103 of the expandable strut 102 at a proximal end 111 of the first cap 110. In some embodiments, a first cap may be integral with a strut to form a monolithic body that in whole or in part forms the vertebral implant. In the embodiment shown, the first end 103 of the expandable strut 102 includes peg holes 107 into which pegs 170 may be inserted to secure the first cap 110 to the expandable strut 102. The first cap 110 shown includes a first wall 115 extending between the proximal end 111 of the first cap 110 and the distal end 112 of the first cap 110. The illustrated first wall 115 is a substantially circular wall in cross-section that forms a continuous lateral enclosure. In other embodiments, a first wall may be formed in combination with other walls or wall components or singularly form a full or partial lateral enclosure of any functional shape. For example and without limitation, the lateral periphery of one or more walls may be substantially an oval, kidney shape, triangle, rectangle, square, any polygonal or curved shape, or any combination of shapes. Embodiments of the first wall include materials that substantially block the transmission or radiation through the first wall.

The first wall 115 illustrated in FIG. 5 may include one or both of structural components and radiation blocking materials, at least as described in association with the first wall 15 herein. The structural components of various embodiments may also be capable of blocking the transmission of radiation. The structural component of some embodiments does not significantly block the transmission of radiation and may in these embodiments be referred to as a radiolucent material. For example and without limitation, a radiolucent structural component may be made from PEEK material. The first wall 115 may be made from a material that substantially blocks the transmission of radiation through the first wall 115. Non-limiting example materials of which the first wall 115 may be made that block the transmission of radiation include cobalt chrome, titanium, and stainless steel. A radiation blocking material 117 may be applied to the inside or outside or be encapsulated within a wall such as the first wall 115. The radiation blocking material 117 is shown applied to the inside of the first wall 115. Materials of which the radiation blocking material 117 may be made or may include are, by way of example and without limitation, gold, lead, barium, bismuth, tantalum, tin, and tungsten. A radiation blocking material may be applied to or integrated with a wall embodiment by any effective mechanism, including but not limited to, chemically bonding, an intervening adhesive, welding, melting, press fitting, ion deposition, or mechanically locking.

As shown in FIG. 5, a first floor 118 extends from the first wall 115. The illustrated first wall 115 and the first floor 118 form a cavity 140 near the distal end 112 of the first cap 110. In the illustrated embodiment, the first wall 115 forms a continuous lateral enclosure and the first floor 118 connects with first wall 115 around the entire periphery of the first cap 110. In other embodiments, the first floor 118 may connect with the first wall 115 in part and with other components or walls in part. The first floor 118 of the illustrated embodiment blocks the transmission of radiation through the first floor 118 toward the proximal end 111 of the first cap 110. In other embodiments, a first floor may be designed to allow the transmission of a selected amount of radiation through the first floor. The first floor 118 may be similar to the first wall 115 wherein it is made from materials that block the transmission of radiation alone, or the first floor 118 may comprise a structural component in combination with a radiation blocking material such as the radiation blocking material 117.

The vertebral implant 101 illustrated in FIG. 5 includes a ceiling 150 extending across at least a portion of the distal end 112 of the first cap 110. The ceiling 150 shown in partial cross-section is a solid material. Spikes 154 are included on the ceiling at the distal end 112 of the first cap 110 that may be pushed into a vertebra to secure the vertebral implant 101 to a vertebra. In other embodiments, teeth, knurling, roughening, ridges, indents, a dome or similar convex shape of the entire ceiling, or any other functional structure may be used to improve engagement between a vertebral implant and a vertebra. The ceiling 150 may be constructed from radiolucent material. In some embodiments, radiation emitting materials may be physically contained, but resulting radiation may be permitted to pass from the distal end 112 through the ceiling 150 to treat tissues.

In some embodiments, the ceiling 150 is configured to support load transferred from an endplate of the first vertebra to the first cap 110. The ceiling 150 may be configured to support load transferred from an endplate of the first vertebra when the ceiling 150 is thick enough to provide resistance in bending and its connections to the first wall 115 are sufficient to support loading, rather than merely to be deflected and to allow substantially all of the load to bear on the first wall 115. In the illustrated embodiment, the ceiling 150 extends across the distal end 112 of the first cap 110 to enclose the cavity 140 and distal end 112. In other embodiments, the ceiling 150 may only in part enclose or cover the distal end 112.

The second cap 130 is shown coupled to the second end 104 of the strut 102. The second cap 130 may include a proximal end 131 coupled to the second end 104 of the strut 102 and a distal end 132 configured to engage with the second vertebra. In some embodiments, a second cap may be integral with a strut to form a monolithic body that in whole or in part forms the vertebral implant. The second cap 130 may also include a second wall 135 extending between the proximal end 131 of the second cap 130 and the distal end 132 of the second cap 130. The second wall 135 of some embodiments includes materials that substantially block the transmission of radiation through the second wall 135. Materials and positioning of the second wall 135 and a second floor (not shown), adjacent to the proximal end 131, and related components may be substantially similar to the materials and positioning of the first wall 115 and the first floor 118 describe in detail herein.

The vertebral implant 101 may also include a radiation emitting device such as the radiation emitting device 1000 illustrated in FIG. 3 or any other functional radiation emitting device. A radiation emitting device may be placed in one or both of the first cap 110 and the second cap 130 as similarly described in association with the first cap 10 herein.

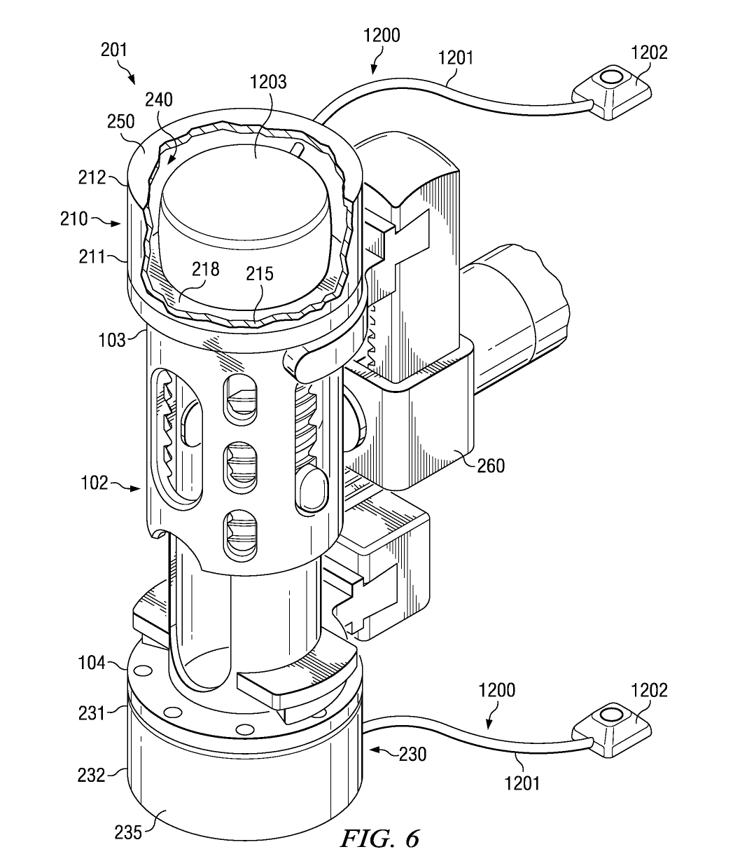

The vertebral implant 201 and a portion of an insertion instrument 260 are illustrated in FIG. 6. The vertebral implant 201 shown includes the expandable strut 102, a first cap 210, and a second cap 230. The illustrated first cap 210 is shown in a partially cut-away view to better illustrate some components. The vertebral implant 201 may be sized to contact a first vertebra adjacent to a space left by one or more central vertebra removed from a spinal column, and also sized to contact a second vertebra adjacent to the space left by one or more central vertebra removed from a spinal column. The vertebral implant 201 may be sized to provide support between the first vertebra and the second vertebra. The strut 102 is illustrated in a substantially expanded state. Features and variations of the strut 102 are described in greater detail in association with FIG. 5.

The first cap 210 of the illustrated embodiment is shown coupled to the first end 103 of the expandable strut 102 at a proximal end 211 of the first cap 210. The first cap 210 may be coupled with the first end 103 of the expandable strut 102 with pegs and holes, as shown in FIG. 5, or by any effective device, including being integrally formed with the expandable strut 102. The first cap 210 shown includes a first wall 215 extending between the proximal end 211 of the first cap 210 and a distal end 212 of the first cap 210. The illustrated first wall 215 is a substantially circular wall in cross-section that forms a continuous lateral enclosure. In other embodiments, a first wall may be formed in combination with other walls or wall components or singularly form a full or partial lateral enclosure of any functional shape. For example and without limitation, the lateral periphery of one or more walls may be substantially an oval, kidney shape, triangle, rectangle, square, any polygonal or curved shape, or any combination of shapes. Embodiments of the first wall include materials that substantially block the transmission or radiation through the first wall.

The first wall 215 illustrated in FIG. 6 may include one or both of structural components and radiation blocking materials, at least as described in association with the first wall 15 herein.

As shown in FIG. 6, a first floor 218 extends from the first wall 215. The illustrated first wall 215 and the first floor 218 form a cavity 240 near the distal end 212 of the first cap 210. In the illustrated embodiment, the first wall 215 forms a continuous lateral enclosure and the first floor 218 connects with first wall 215 around the entire periphery of the first cap 210. In other embodiments, the first floor 218 may connect with the first wall 215 in part and with other components or walls in part. The first floor 218 of the illustrated embodiment blocks the transmission of radiation through the first floor 218 toward the proximal end 211 of the first cap 210. In other embodiments, a first floor may be designed to allow the transmission of a selected amount of radiation through the first floor. The first floor 218 may be similar to the first wall 215 wherein it is made from materials that block the transmission of radiation alone, or the first floor 218 may comprise a structural component in combination with a radiation blocking material such as the radiation blocking material 17.

The vertebral implant 201 illustrated in FIG. 6 includes a ceiling 250 extending across at least a portion of the distal end 212 of the first cap 210. The ceiling 250 shown in partial cross-section is a solid material. Any effective structure to improve engagement between a vertebral implant and a vertebra may be included on the ceiling 250, facing an adjacent vertebra. By way of non-limiting example, the structures to improve engagement may include spikes, teeth, knurling, roughening, ridges, indents, a dome or similar convex shape of the entire ceiling, or any other functional structure. The ceiling 250 may be constructed from radiolucent material. In some embodiments, radiation emitting materials may be physically contained, but resulting radiation may be permitted to pass from the distal end 212 through the ceiling 250 to treat tissues.

In some embodiments, the ceiling 250 is configured to support load transferred from an endplate of the first vertebra to the first cap 210. The ceiling 250 may be configured to support load transferred from an endplate of the first vertebra when the ceiling 250 is thick enough to provide resistance in bending and its connections to the first wall 215 are sufficient to support loading, rather than merely to be deflected and to allow substantially all of the load to bear on the first wall 215. In the illustrated embodiment, the ceiling 250 extends across the distal end 212 of the first cap 210 to enclose the cavity 240 and the distal end 212. In other embodiments, the ceiling 250 may only in part enclose or cover the distal end 212.

The second cap 230 is shown coupled to the second end 104 of the strut 102. The second cap 230 may include a proximal end 231 coupled to the second end 104 of the strut 102 and a distal end 232 configured to engage with the second vertebra. In some embodiments, a second cap may be integral with a strut to form a monolithic body that in whole or in part forms the vertebral implant. The second cap 230 may also include a second wall 235 extending between the proximal end 231 of the second cap 230 and the distal end 232 of the second cap 230. The second wall 235 of some embodiments includes materials that substantially block the transmission of radiation through the second wall 235. Materials and positioning of the second wall 235 and a second floor (not shown), adjacent to the proximal end 231, and related components may be substantially similar to the materials and positioning of the first wall 215 and the first floor 218 describe in detail herein.

The vertebral implant 201 may also include a radiation emitting device such as the radiation emitting device 1000 illustrated in FIG. 3 and effective variations. A radiation emitting device of any effective type may be placed in one or both of the first cap 210 and the first cap 230 as similarly described for the first cap 10 herein. In FIG. 6, radiation emitting devices 1200 are illustrated in both the first cap 210 and the second cap 230. The radiation emitting device 1200 is a GliaSite® Catheter available from Proxima Therapeutics, Inc. of Alpharetta, Ga. A GliaSite® Catheter may include a radiopaque silicone tube 1201, a low-profile infusion port 1202 at a proximal end, and a silicone balloon 1203 at a distal end. Radiation may be applied by injecting or otherwise delivering a radioactive substance through the infusion port 1202. For example, Iotrex® may be delivered through the infusion port 1202, then through the silicone tube 1201, and finally into the silicone balloon 1203. In order to regulate the amount of radiation delivered to a treatment area, the radioactive substance may be one or both of withdrawn from the balloon 1203 and allowed to decay over a period of time.

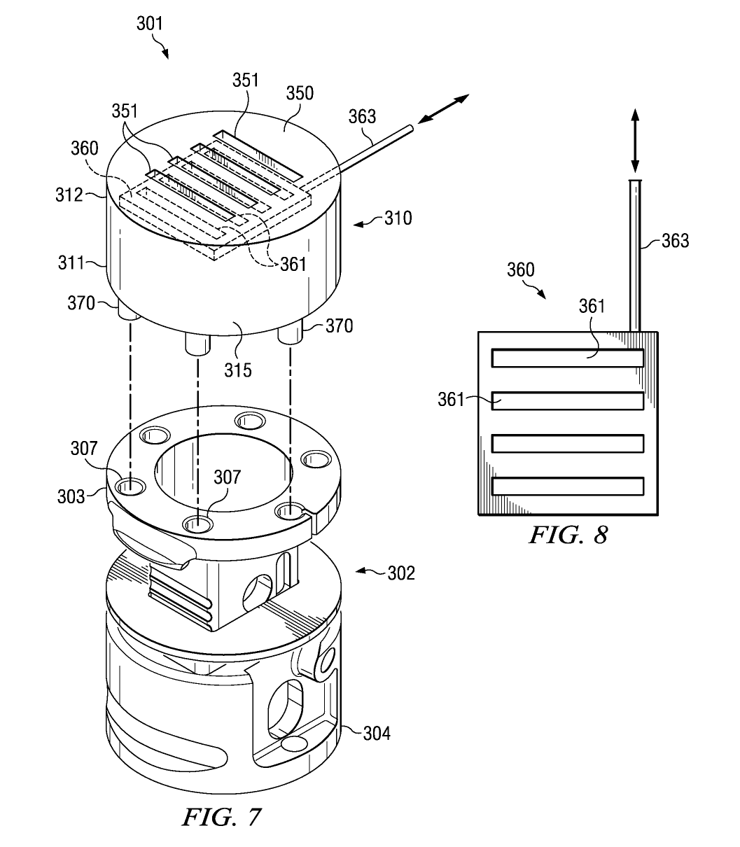

The vertebral implant 301 is illustrated in FIG. 7, and includes an expandable strut 302 and a first cap 310. The vertebral implant 301 may be sized to contact a first vertebra adjacent to a space left by one or more central vertebra removed from a spinal column, and also sized to contact a second vertebra adjacent to the space left by one or more central vertebra removed from a spinal column. The vertebral implant 301 may be sized to provide support between the first vertebra and the second vertebra.

The strut 302 shown is a T2 XVBR™ Spinal Implant available from Medtronic, Inc. and includes a first end 303 and a second end 304. The expandable strut 302 is illustrated in a partially contracted state with portions of the first end 303 nested within the second end 304. The expandable strut 302 may include titanium material and may be expandable by ratcheting or sliding and locking between components of the first and second ends 303, 304. In addition or as an alternative to titanium, the expandable strut 302 may be made from any biocompatible material. For example and without limitation, the expandable strut 302 may be made in whole or in part from polyetheretherketone (PEEK), cobalt chrome, stainless steel, and any biocompatible metal, metal alloy, or polymer. The expandable strut 302 may also include a bone or bone-based material. For example and without limitation, the expandable strut 302 may include in whole or in part one or more of allograft, xenograft, demineralized bone, and autograft.

The first cap 310 of the illustrated embodiment is shown in an exploded position relative to the expandable strut 302 and aligned to be coupled to the first end 303 of the expandable strut 302 at a proximal end 311 of the first cap 310. In the embodiment shown, the first end 303 of the expandable strut 302 includes peg holes 307 into which pegs 370 may be inserted to secure the first cap 310 to the expandable strut 302. In some embodiments, a first cap may be integral with a strut to form a monolithic body that in whole or in part forms the vertebral implant. The first cap 310 shown includes a first wall 315 extending between the proximal end 311 of the first cap 310 and the distal end 312 of the first cap 310. The illustrated first wall 315 is a substantially circular wall in cross-section that forms a continuous lateral enclosure. In other embodiments, a first wall may be formed in combination with other walls or wall components or singularly form a full or partial lateral enclosure of any functional shape. For example and without limitation, the lateral periphery of one or more walls may be substantially an oval, kidney shape, triangle, rectangle, square, any polygonal or curved shape, or any combination of shapes. Embodiments of the first wall include materials that substantially block the transmission or radiation through the first wall.

The first wall 315 illustrated in FIG. 7 may include one or both of structural components and radiation blocking materials, at least as described in association with the first wall 15 herein.

A first floor (not shown), adjacent to the proximal end 311, may extend from the first wall 315 similarly to first floors 18, 118, 218 described and illustrated herein. The illustrated first wall 315 and the first floor may form a cavity near the distal end 312 of the first cap 310. In the illustrated embodiment, the first wall 315 forms a continuous lateral enclosure and the first floor connects with first wall 315 around the entire periphery of the first cap 310. In other embodiments, the first floor may connect with the first wall 315 in part and with other components or walls in part. The first floor may block the transmission of radiation through the first floor toward the proximal end 311 of the first cap 310. In some embodiments, a first floor may be designed to allow the transmission of a selected amount of radiation through the first floor. The first floor may be similar to the first wall 315 wherein it is made from materials that block the transmission of radiation alone, or the first floor may comprise a structural component in combination with a radiation blocking material.

The vertebral implant 301 illustrated in FIG. 7 includes a ceiling 350 extending across at least a portion of the distal end 312 of the first cap 310. In some embodiments, radiation emitting materials may be physically contained, but resulting radiation may be permitted to pass from the distal end 312 through the ceiling 350 to treat tissues. The ceiling 350 shown may be actuated to change between a state that substantially blocks the transmission of radiation through the ceiling 350 and a state that substantially allows the transmission of radiation through the ceiling 350. In particular, the illustrated ceiling 350 includes materials that substantially block the transmission of radiation, but also includes slots 351 through which radiation may be transmitted.

A plate 360 is shown in FIGS. 7 and 8. The illustrated plate 360 is constructed of a material or materials that substantially block the transmission of radiation, but includes plate slots 361 through which radiation may be transmitted. In the state illustrated in FIG. 7, the slots 351 are aligned with solid portions of the plate 360 and the plate slots 361 are aligned with solid portions of the ceiling 350. Therefore, radiation is substantially blocked from transmission through the ceiling 350. Another state is achieved by sliding the plate 360 relative to the ceiling 350 such that the slots 351 and the plate slots 361 are aligned with one another to substantially allow the transmission of radiation through the ceiling 350. The plate 360 may be moved relative to the ceiling 350 by pushing and pulling on an actuator 363. Other actuators may include, but are not limited to, pairs of wires, threaded shafts, and magnetically, electrically, and hydraulically responsive devices. An actuator may extend transcutaneously from a patient such that the transmission of radiation through a ceiling may be altered periodically from an action outside of the body of a patient. An actuator may also be subcutaneous, but reachable for use by a medical professional. Any other effective mechanism for switching between a state that allows transmission of radiation through the ceiling 350 and a state that blocks transmission of radiation through the ceiling 350 may be used. For example and without limitation, rotary mechanisms, a shutter type opening, and mechanisms that separate a plate from the ceiling may be used.

Any effective structure to improve engagement between a vertebral implant and a vertebra may be included on the ceiling 350, facing an adjacent vertebra. By way of non-limiting example, the structures to improve engagement may include spikes, teeth, knurling, roughening, ridges, indents, a dome or similar convex shape of the entire ceiling, or any other functional structure. The ceiling 350 may be constructed from radiolucent material.

In some embodiments, the ceiling 350 is configured to support load transferred from an endplate of the first vertebra to the first cap 310. The ceiling 350 may be configured to support load transferred from an endplate of the first vertebra when the ceiling 350 is thick enough to provide resistance in bending and its connections to the first wall 315 are sufficient to support loading, rather than merely to be deflected and to allow substantially all of the load to bear on the first wall 315. In the illustrated embodiment, the ceiling 350 extends across the distal end 312 of the first cap 310 to enclose a cavity at the distal end 312. In other embodiments, the ceiling 350 may only in part enclose or cover the distal end 312.

In some embodiments, a second cap may be coupled to the second end 304 of the strut 302. A second cap of the vertebral implant 301 may be integral with a strut to form a monolithic body that in whole or in part forms the vertebral implant 301. The second cap may engage with the second vertebra.

The vertebral implant 301 may also include a radiation emitting device such as the radiation emitting devices 1000, 1200 illustrated in FIG. 3 and 6 respectively. A radiation emitting device may be placed in one or both of the first cap 310 and a second cap (not shown), as similarly described for the first caps 10, 210 and second cap 230 herein.

Embodiments of the device may be further described as means for applying radiation to a vertebral body. In particular, a device may include a spacing means for providing support between two vertebral bodies as shown for struts 2, 102, 302, and described in combination with a radiation emitting device coupled to the spacing means. The spacing means may include a radiation containment means that is capable of blocking transmission of radiation from the radiation emitting device in one or more directions and permitting transmission of radiation from the radiation emitting device in one or more directions. In particular, a corpectomy or vertebrectomy device may provide support between vertebrae and include features that direct radiation toward vertebrae or portions of vertebrae to which the corpectomy or vertebrectomy devices are coupled.

Any of the struts or caps described above may be filled in whole or in part with an osteogenic material or therapeutic composition. Osteogenic materials include, without limitation, autograft, allograft, xenograft, demineralized bone, synthetic and natural bone graft substitutes, such as bioceramics and polymers, and osteoinductive factors. A separate carrier to hold materials within the device may also be used. These carriers may include collagen-based carriers, bioceramic materials, such as BIOGLASS®, hydroxyapatite and calcium phosphate compositions. The carrier material may be provided in the form of a sponge, a block, folded sheet, putty, paste, graft material or other suitable form. The osteogenic compositions may include an effective amount of a bone morphogenetic protein (BMP), transforming growth factor β1, insulin-like growth factor, platelet-derived growth factor, fibroblast growth factor, LIM mineralization protein (LMP), and combinations thereof or other therapeutic or infection resistant agents, separately or held within a suitable carrier material.

Embodiments of the implant in whole or in part may be constructed of biocompatible materials of various types. Examples of implant materials include, but are not limited to, non-reinforced polymers, carbon-reinforced polymer composites, PEEK and PEEK composites, low density polyethylene, shape-memory alloys, titanium, titanium alloys, cobalt chrome alloys, stainless steel, ceramics and combinations thereof. In some embodiments, the implant or individual components of the implant may be constructed of solid sections of bone or other tissues. Tissue materials include, but are not limited to, synthetic or natural autograft, allograft or xenograft, and may be resorbable or non-resorbable in nature. Examples of other tissue materials include, but are not limited to, hard tissues, connective tissues, demineralized bone matrix and combinations thereof.

FIG. 1 illustrates four vertebrae, V₁-V₄, of a typical lumbar spine and three spinal discs, D₁-D₃. Embodiments of the invention may be applied to the lumbar spinal region, and embodiments may also be applied to the cervical or thoracic spine or between other skeletal structures.

Some embodiments may also include supplemental fixation devices in addition to or as part of the expandable medical implant for further stabilizing the anatomy. For example, and without limitation, rod and screw fixation systems, anterior, posterior, or lateral plating systems, facet stabilization systems, spinal process stabilization systems, and any devices that supplement stabilization may be used as a part of or in combination with the expandable medical implant. Embodiments of the invention may be useful in at least some spinal fusion procedures where a spinal disc is replaced without replacing a vertebral body.

An embodiment of the invention is a method of selectively irradiating tissues in and around a spinal column. In particular, a method may include irradiating cells of all or part of a treatable vertebral body. The method may also include removing all or a portion of a central vertebral body that is adjacent to the treatable vertebral body. A vertebral implant or vertebral body replacement implant, that is a substitute for a vertebral body or a portion of a vertebral body, such as any of the vertebral implants 1, 101, 201, 301, may be provided. A provided vertebral body implant of some embodiments continuously or selectively substantially blocks the transmission of radiation in all directions except the direction toward the treatable vertebral body when the vertebral body replacement implant is in position in a spinal column. The implant may also include a cavity in which a radiation emitting device may be placed.

An additional act of various method embodiments is to insert a radiation emitting device in the cavity in the vertebral body replacement implant. The radiation emitting device may be inserted at any time during the process of acting on the treatable vertebral body. For example and without limitation, all or a part of the radiation emitting device may be inserted into the cavity in the vertebral body replacement implant prior to placement of the implant into a patient. Alternatively or in addition, the radiation emitting device or component parts of the radiation emitting device may be inserted the implant after it is in place in a patient or partially in place in a patient. The radiation emitting device or components of the radiation emitting device may be inserted one or more of pre-operatively, inter-operatively, and post-operatively. The radiation emitting device may be a device capable of receiving radiation or components that emit radiation and may not at all times be able to emit radiation. That is, its designation as an “emitting device” does not mean that it, or one or more of its component parts, are at all times capable of emitting radiation.

In another act of various method embodiments of the invention, the vertebral body replacement implant is implanted in a spinal column. In some devices, radiation is emitted from the vertebral body replacement implant with no further acts. In other devices, additional material must be introduced into the vertebral body replacement implant. For example and without limitation, the radiation emitting device illustrated in FIG. 6 may require the introduction of a radioactive material, such as Iotrex®, through the infusion port 1202 and into the vertebral implant, as described herein. With some devices, the vertebral body replacement implant must be actuated to allow transmission of radiation toward the treatable vertebral body. For example and without limitation, the vertebral implant depicted in FIGS. 7 and 8 illustrates a device that is actuated to change between a state that substantially blocks the transmission of radiation toward the treatable vertebral body, when the slots 351 and the plate slots 361 do not at least in part align, and a state that substantially allows the transmission of radiation toward the treatable vertebral body, when the slots 351 and the plate slots 361 do at least in part align.

Embodiments of the vertebral implant may be implanted from any surgical approach, including but not limited to, posterior, lateral, anterior, transpedicular, lateral extracavitary, in conjunction with a laminectomy, in conjunction with a costotransversectomy, or by any combination of these and other approaches.

Various method embodiments of the invention are described herein with reference to particular vertebral implants. However, in some circumstances, each disclosed method embodiment may be applicable to each of the implants, or to some other implant operable as disclosed with regard to the various method embodiments.

Terms such as anterior, posterior, lateral, proximal, distal, side, and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A vertebral implant for replacing at least an anterior portion of a central vertebra and at least portions of two spinal discs, wherein the at least anterior portion of the central vertebra is removed from between a first vertebra and a second vertebra, the vertebral implant comprising:
a strut having a first end and an opposite second end; and
a first cap coupled to the first end of the strut, the first cap comprising:
a proximal end coupled to the first end of the strut,
a distal end configured to engage with the first vertebra,
a first wall extending between the proximal end of the first cap and the distal end of the first cap, wherein the first wall includes materials that substantially block the transmission of radiation through the first wall, and
a first floor extending from the first wall, wherein the first floor and the first wall form a cavity near the distal end of the first cap;
wherein the vertebral implant is adapted to contact the first vertebra and the second vertebra and provide support between the first vertebra and the second vertebra.

2. The vertebral implant of claim 1 wherein the strut includes a mesh body.

3. The vertebral implant of claim 1 wherein the strut comprises bone material.

4. The vertebral implant of claim 1 wherein the strut is an expandable strut configured to be expanded from a first height to a second taller height.

5. The vertebral implant of claim 1 wherein the first wall is made from a material that substantially blocks the transmission of radiation through the first wall.

6. The vertebral implant of claim 1 wherein the first wall comprises a radiolucent material in combination with a material that substantially blocks the transmission of radiation through the first wall.

7. The vertebral implant of claim 1 wherein the first floor includes materials that substantially block the transmission of radiation through the first floor toward the proximal end of the first cap.

8. The vertebral implant of claim 1, further comprising a ceiling extending across at least a portion of the distal end of the first cap.

9. The vertebral implant of claim 8 wherein the ceiling comprises a radiolucent material.

10. The vertebral implant of claim 8 wherein the ceiling may be actuated to change between a state that substantially blocks the transmission of radiation through the ceiling and a state that substantially allows the transmission of radiation through the ceiling.

11. The vertebral implant of claim 1, further comprising a ceiling extending across the first cap that encloses the cavity.

12. The vertebral implant of claim 11 wherein the ceiling is adpated to support load transferred from an endplate of the first vertebra to the first cap.

13. The vertebral implant of claim 11 wherein the ceiling comprises a radiolucent material.

14. The vertebral implant of claim 11 wherein the ceiling may be actuated to change between a state that substantially blocks the transmission of radiation through the ceiling and a state that substantially allows radiation to be transmitted through the ceiling.

15. The vertebral implant of claim 1, further comprising a radiation emitting device coupled to the first cap.

16. The vertebral implant of claim 1, further comprising a second cap coupled to the second end of the strut, the second cap comprising:
a proximal end coupled to the second end of the strut,
a distal end configured to engage with the second vertebra, and
a second wall extending between the proximal end of the second cap and the distal end of the second cap, wherein the second wall includes materials that substantially block the transmission of radiation through the second wall.

17. The vertebral implant of claim 16, further comprising a second floor extending from the second wall, wherein the second floor and the first wall form a cavity near the distal end of second first cap.

18. The vertebral implant of claim 17 wherein the second floor includes materials that substantially block the transmission of radiation through the second floor toward the proximal end of the second cap

19. The vertebral implant of claim 16, further comprising a radiation emitting device coupled to the second cap.

20. A means for applying radiation to a vertebral body comprising:
a spacing means for providing support between two vertebral bodies; and
a radiation emitting device coupled to the spacing means;
wherein the spacing means includes a radiation containment means that is capable of blocking transmission of radiation from the radiation emitting device in one or more directions and permitting transmission of radiation from the radiation emitting device in one or more directions.

21. A method of irradiating cells of a treatable vertebral body in a spinal column comprising:
removing a central vertebral body that is adjacent to the treatable vertebral body;
providing a vertebral body replacement implant with a cavity, wherein the vertebral body replacement implant substantially blocks the transmission of radiation in all directions except the direction toward the treatable vertebral body when the vertebral body replacement implant is implanted;
inserting a radiation emitting device in the cavity in the vertebral body replacement implant; and
implanting the vertebral body replacement implant in a spinal column.

22. The method of claim 21, further comprising actuating the vertebral body replacement implant to change between a state that substantially blocks the transmission of radiation toward the treatable vertebral body and a state that substantially allows the transmission of radiation toward the treatable vertebral body.
